(19) United States
(12) Patent Application Publication
(10) Pub. No.: US 2019/0392955 A1
(43) Pub. Date: Dec. 26, 2019

(54) SYSTEM AND METHOD FOR DRUG INTERACTION PREDICTION

(71) Applicant: MedAware Ltd., RaAnana (IL)

(72) Inventor: Eitan ISRAELI, Reut (IL)

(73) Assignee: MedAware Ltd., RaAnana (IL)

(21) Appl. No.: 16/478,495

(22) PCT Filed: Mar. 8, 2018
(86) PCT No.: PCT/IL2018/050272

§ 371 Date: Jul. 17, 2019

Related U.S. Application Data
(60) Provisional application No. 62/468,392, filed on Mar. 8, 2017.

(51) Int. Cl. G16H 70/40; G16H 20/10; G16H 10/60; G06F 17/18; G06F 16/2457

(52) U.S. Cl. CPC G16H 70/40; G16H 20/10; G06F 16/2457; G06F 17/18; G16H 10/60

(57) ABSTRACT

A system for drug interaction alerts and a method using same are provided. The system includes a computing platform configured for obtaining prescribing history for each of a drug A and a drug B and for drug A and drug B from medical records of a patient cohort. The system is further configured for determining a statistical probability for co-prescribing drug A and drug B versus a product of the statistical probability for prescribing drug A and the statistical probability for prescribing drug B, under different clinical contexts. This probability is then used to indicate a likelihood of drug interaction in a subject.

### FIELD AND BACKGROUND OF THE INVENTION

The present invention relates to a system and method for assessing the likelihood that a drug combination can be prescribed to a subject, and more particularly, to a system that provides pharmacists and physicians with more accurate and subject-specific information regarding the suitability of use of a drug combination.

Drug-drug interactions (DDIs) are a significant cause of patient morbidity and mortality and can significantly increase hospitalization costs.

Commercial software programs designed to assist pharmacists during prescription processing and physicians during prescribing have included DDI alerts for many years. However, the large number of false alerts issued by these systems causes user desensitization and alert fatigue leading pharmacists and physicians to disregard true alerts in many cases.

Due to these limitations, currently used DDI systems do not achieve their intended goal. Attempts at solving this problem have centered on reducing the number of alerts by reclassifying interactions based on severity and historical rate of acceptance, as well as expert opinions. However, to date DDI systems are still considered ineffective due to the aforementioned problems and are ignored by many physicians and pharmacists.

There is thus a need for, and it would be highly advantageous to have, a DDI system or a DDI-adjunct system devoid of the above limitations.

### SUMMARY OF THE INVENTION

According to one aspect of the present invention there is provided a system for drug interaction alerts comprising a computing platform configured for: (a) obtaining prescribing history for each of a drug A and a drug B from medical records of a patient cohort; (b) obtaining co-prescribing history for drug A and drug B from the medical records of the patient cohort; (c) determining a statistical probability for co-prescribing drug A and drug B [Prob (A and B)] versus a product of the statistical probability for prescribing drug A and the statistical probability for prescribing drug B [Prob(a)×Prob(B)]; and (d) indicating a low likelihood of drug interaction if [Prob (A and B)] divided by [Prob(a)×Prob(B)] is above a predetermined threshold.

According to further features in preferred embodiments of the invention described below, (d) is provided in response to a desired drug interaction alert frequency.

According to still further features in the described preferred embodiments the predetermined threshold is a function of a desired drug interaction alert severity (clinical significance of alert) provided by a drug interaction database.

According to still further features in the described preferred embodiments the patient cohort is defined by at least one clinical indication.

According to still further features in the described preferred embodiments a patient cohort is formed around a clinical indication determined via machine learning analysis of a patient population.

According to still further features in the described preferred embodiments the at least one clinical indication is derived from blood test results, a prescribing history, a diagnosis, a treatment and/or a physiological parameter.

According to still further features in the described preferred embodiments the medical records are derived from one or more electronic medical records databases.

According to another aspect of the present invention there is provided a method of assessing for a subject a likelihood of drug interaction comprising: (a) obtaining prescribing history for each of a drug A and a drug B from medical records of a patient cohort; (b) obtaining co-prescribing history for drug A and drug B from the medical records of the patient cohort; (c) determining a statistical probability for co-prescribing drug A and drug B [Prob (A and B)] versus a product of the statistical probability for prescribing drug A and the statistical probability for prescribing drug B [Prob(a)×Prob(B)]; and (d) indicating a low likelihood of drug interaction in the subject if [Prob (A and B)] divided by [Prob(a)×Prob(B)] is above a predetermined threshold.

According to still further features in the described preferred embodiments (d) is provided in response to a desired drug interaction alert severity.

According to still further features in the described preferred embodiments the predetermined threshold is a function of a number of drug interaction alerts.

According to still further features in the described preferred embodiments the patient cohort shares at least one clinical indication with the subject.

According to still further features in the described preferred embodiments a patient cohort is formed around a clinical indication determined via machine learning analysis of a patient population.

According to still further features in the described preferred embodiments the at least one clinical indication is derived from blood test results, a prescribing history, a diagnosis, a treatment and/or a physiological parameter.

According to still further features in the described preferred embodiments the medical records are derived from one or more electronic medical records databases.

The present invention successfully addresses the shortcomings of the presently known configurations by providing a drug interaction system which can issue subject-specific drug interaction alerts or verify drug interaction alerts issued by another drug interaction system.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a block diagram illustrating the present system.

FIGS. 2A-B are flowcharts illustrating the steps of the present approach.

### DESCRIPTION OF THE PREFERRED EMBODIMENTS

The present invention is of a system which can be used to provide drug interaction alerts or verify drug interaction alerts issued by another drug interaction alert system. Specifically, the present invention can be used to determine if a drug-drug interaction alert triggered under certain circumstances is accurate enough to be presented to the physician while taking into account the “alert fatigue” effect incurred by false alarms.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Most current DDI systems use large drug-interaction databases to identify potentially hazardous drug combinations. The usual format of these databases is: Drug-A, Drug-B, severity (low, medium, high). These databases contain about ˜200K drug pair combinations.

Healthcare providers and pharmacists are shown an alert whenever drug A and drug B are co-prescribed, according to a pre-defined severity threshold. In order to reduce false alarm rate, current systems allow users to define the minimal severity threshold for alerts as well as to manually remove individual drug combinations which are presumed to generate false alarms. However, the alarm rate of these systems is somewhere between 7% and 30% for all prescriptions and the false-alarm rate is often higher than 90% even with high severity settings. This causes “alert fatigue” which leads physicians/pharmacists to ignore alerts. Reducing such alert fatigue by selectively presenting only the most relevant alerts would increase physician response to the alerts and would significantly decrease the overall risk of true DDI events.

In efforts of reducing alert fatigue and physician/pharmacist desensitization, the present inventors devised an alert system, which utilizes medical records of a specific cohort of patients (of a population of patients) matched with a subject of interest in order to assess the potential relevance of a specific drug interaction alert.

As is further described herein, the present system can be used as a standalone alert system or as a verification system for commercially available DDI systems.

Thus, according to one aspect of the present invention there is provided a system for drug interaction alerts or alert verification.

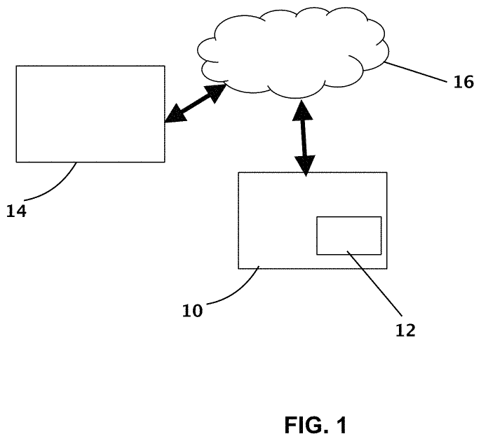

FIG. 1 illustrates the present system which is referred to herein as system 10. System 10 includes a computing platform 12 configured for obtaining prescribing history for each of a drug A and a drug B from medical records of a patient population (from an EMR database 14) and obtaining co-prescribing history for drug A and drug B from the medical records of the patient population. EMR database can be integrated into system 10 or linked thereto via a communication network (16 in FIG. 1).

The medical records of the patient cohort can be electronic medical records (EMR) available from EMR systems such as Meditech, Cerner, Epic systems and the like, or they can be obtained from personal health records applications such as My Medical, Track My Medical Records and the like, or medical claims data from pharmacies, Pharmacy Benefit Management companies (PBMs) and health plans.

An electronic medical record is a digital version of the paper file used in a physician's office or clinic. The EMR contains the medical history of a patient including demographics, office visits, diagnosis, procedures, prescriptions, laboratory and examination results longitudinally listed over time.

The medical records are processed via a processing unit of the present system to construct a database of drug prescriptions and co-prescriptions associated with specific medical conditions/indications. The database determines when (i.e. under what clinical condition) co-prescriptions are relatively common and as such potentially safe, or when co-prescriptions are rare and thus potentially unsafe. Co-prescriptions in subjects having specific medical conditions should be alerted upon if the statistical probability for co-prescribing drug A and drug B [Prob (A and B)] is much smaller than the statistical probability for prescribing drug A multiplied by the statistical probability for prescribing drug B [Prob(a)×Prob(B)] for a specific medical condition.

Thus, the database of the present system is constructed from EMR of a heterogeneous patient population or from a patient cohort (subgroup of population) characterized by at least one parameter (condition/indication/patient history) and defines a range of clinical conditions and alert settings (when an alert is triggered and when not) for each condition and subject.

The database can be constructed via machine learning using a classification algorithms (e.g. Random Forest, Support Vector Machine) to identify (for a pair of drugs) significant clinical indicators, and combinations of indicators indicating when co-prescribing should trigger an alert or not.

The present system utilizes the database to provide a user with an indication of a low likelihood of drug interaction if:

[Prob (A and B)] divided by [Prob(a)×Prob(B)]

is below a predetermined threshold [also referred to hereinunder as “false alarm likelihood score” (FALS)].

The present system can either confirm a DDI alert provided by a standard DDI system or provide the user with information that can be used to possibly ignore such a DDI alert. In any case, the present system provides the user with additional information that can be useful in making a prescribing decision.

The threshold can be set by the user anywhere from show all to block all alerts, or according to one or more of the following meaningful/useful parameters:

(i) Desired alert frequency—for every 50-1000 prescriptions;

(ii) False alarm rate below 10-25%, based on actual physician response;

(iii) 70-100% alarm rate on potentially hazardous co-prescriptions (severe clinical implications), based on standard tests as LeapFrog™ (www(dot)leapfroggroup(dot)org/ratings-reports/computerized-physician-order-entry; and www(dot)leapfroggroup(dot)org/sites/default/files/Files/CPOE%20Fact%20Sheet (dot)pdf).

(iv) Thus, the present system mines prescribing history for each individual drug, and for each pair of drugs currently in a drug interaction database and identifies various patient cohorts with a shared clinical parameter.

Examples of clinical parameters include, but are not limited to:

(i) gender—a patient cohort in which patients are of a single gender;

(ii) physiology—a patient cohort in which patients have one or more physiological parameters (weight, age, BMI, blood pressure, resting HR etc) that fall within a defined range.

(iii) disorders—a patient cohort in which patients have or have had a specific disorder;

(iv) blood results—a patient cohort in which patients have or have had a specific value or value range for one or more blood-derived tests;

(v) procedures/surgeries/imaging results—a patient cohort in which patients have or have had a specific surgical or non-surgical procedure or an imaging exam (e.g. X-ray, CAT scan, MRI etc.); and

(vi) general condition and age of the subject.

The system then utilizes machine learning to build a statistical model to classify in which cohorts the two drugs are not likely to be co-prescribed.

Whenever a prescribing overlap between two drugs is identified, the system assigns a personalized “false alarm likelihood score” (FALS), based on the machine learning model and the specifics of the subject. The system then enables the user to decide for which combination to provide an alert based on the FALS score and user preferences.

The machine learning model is used to recognize clinically reasonable settings where the condition holds at different levels. The FALS is therefore a function of the clinical setting, and increases as the underlying parameter used for grouping the cohort is weaker.

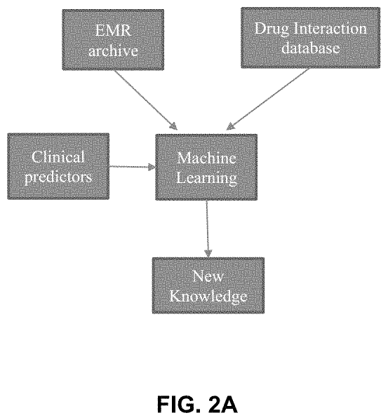

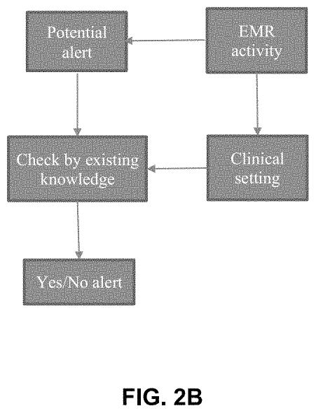

FIGS. 2A-B are flowcharts outlining the learning (FIG. 2A) and execution (FIG. 2B) phases of the present system.

In the learning phase, for every drug and drug combination the system obtains a prescription history from an EMR archive. Based on the EMR information and a comprehensive list of potential clinical predictors (hundreds), a Machine Learning module of the present system constructs a statistical model (“new knowledge”) using a classification algorithms (e.g. Random Forest, Support Vector Machine), that includes a table of drug-drug probabilities (in the form of “drug A and B are not likely to be co-prescribed in a clinical condition X, Y and/or . . . N)”.

This statistical model is then used in real-time to support a DDI alert system (FIG. 2B). The system monitors EMR for any relevant new information (prescriptions, clinical data) for a specific subject. When two drugs are co-prescribed to the subject, the system utilizes the statistical model to determine the likelihood of drug interaction for the subject and provide an indication accordingly (as mainline or adjunct to a standard DDI alert system).

The above can be exemplified as follows: assuming that drug A and drug B should not be given together according to a standard DDI system. If the statistical model of the present system finds A and B to be strongly negatively correlated, then the present system would support an interaction alerts issued by a standard DDI alert system. However, if A and B are strongly negatively correlated only in a specific subset of patients (having a specific clinical condition), then an alert is only supported by the present system in a subject belonging to this subset of patients and not in co-prescribed subjects belonging to other subsets.

While the present system can be used as a standalone drug interaction system, it is typically used along with a standard DDI system to filter DDI warnings presented to the user in order to decrease alert fatigue. In that respect, the present system is a tool layered on top of a DDI system.

As is mentioned hereinabove, the present system can be set anywhere between blocking all alerts and showing all alerts depending on user preferences such as desired precision, alert frequency and the like.

As used herein the term “about” refers to ±10%.

Additional objects, advantages, and novel features of the present invention will become apparent to one ordinarily skilled in the art upon examination of the following examples, which are not intended to be limiting.

### EXAMPLES

Reference is now made to the following examples, which together with the above descriptions, illustrate the invention in a non limiting fashion.

Co-Prescribing of Aldactone and Trimethoprim

Standard DDI systems (e.g. ePocrates, MicroMedex, FDB etc.) classify the combination of Aldactone and Trimethoprim as “severe interaction”. However, examination of numerous medical records by the present inventor revealed that this combination is often co-prescribed in patients with heart failure and in need of adjunctive antibiotic treatment.

By mining EMR historical data of patients and classifying drug prescriptions and co-prescriptions according to parameters such as diagnoses, disorders, indications etc. (using machine learning and the probability equation described herein), the present system can identify this specific DDI alert as less relevant in heart failure patients in need of antibiotic treatment. Thus, when a subject diagnosed with heart failure and a bacterial infection is prescribed with Aldactone and Trimethoprim and an alert is issued by a standard DDI system, the system of the present invention will indicate to the physician/pharmacists that in this specific subject, this alert may be of limited clinical value or alternatively (based on user preferences) not show the alert to the physician/pharmacists.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for drug interaction alerts comprising a computing platform configured for:
(a) obtaining prescribing history for each of a drug A and a drug B from medical records of a patient cohort;
(b) obtaining co-prescribing history for drug A and drug B from said medical records of said patient cohort;
(c) determining a statistical probability for co-prescribing drug A and drug B [Prob (A and B)] versus a product of said statistical probability for prescribing drug A and said statistical probability for prescribing drug B [Prob(a)×Prob(B)]; and
(d) indicating a low likelihood of drug interaction if [Prob (A and B)] divided by [Prob(a)×Prob(B)] is above a predetermined threshold.

2. The system of claim 1, wherein (d) is provided in response to a desired drug interaction alert frequency.

3. The system of claim 2, wherein said predetermined threshold is a function of a desired drug interaction alert severity provided by a drug interaction database.

4. The system of claim 1, wherein said patient cohort is defined by at least one clinical indication.

5. The system of claim 4, wherein said patient cohort is derived from a patient population via machine learning analysis.

6. The system of claim 4, wherein said at least one clinical indication is derived from blood test results, a prescribing history, a diagnosis, a treatment and/or a physiological parameter.

7. The system of claim 1, wherein said medical records are derived from one or more electronic medical records databases.

8. A method of assessing for a subject a likelihood of drug interaction comprising:
(a) obtaining prescribing history for each of a drug A and a drug B from medical records of a patient cohort;
(b) obtaining co-prescribing history for drug A and drug B from said medical records of said patient cohort;
(c) determining a statistical probability for co-prescribing drug A and drug B [Prob (A and B)] versus a product of said statistical probability for prescribing drug A and said statistical probability for prescribing drug B [Prob(a)×Prob(B)]; and
(d) indicating a low likelihood of drug interaction in the subject if [Prob (A and B)] divided by [Prob(a)×Prob(B)] is above a predetermined threshold.

9. The method of claim 8, wherein (d) is provided in response to a desired drug interaction alert frequency.

10. The method of claim 9, wherein said predetermined threshold is a function of a number drug interaction alerts.

11. The method of claim 10, wherein said patient cohort shares at least one clinical indication with the subject.

12. The method of claim 11, wherein said patient cohort is derived from a patient population via machine learning analysis.

13. The method of claim 11, wherein said at least one clinical indication is derived from blood test results, a prescribing history, a diagnosis, a treatment and/or a physiological parameter.

14. The method of claim 8, wherein said medical records are derived from one or more electronic medical records databases.
